(19) United States
(12) Patent Application Publication
(10) Pub. No.: US 2018/0001080 A1
(43) Pub. Date: Jan. 4, 2018

(54) Electrode Fixing Sleeve

(71) Applicant: BIOTRONIK SE & Co. KG, Berlin (DE)

(72) Inventor: Jens Ulmer, Kuesnacht (CH)

(21) Appl. No.: 15/623,628

(22) Filed: Jun. 15, 2017

(30) Foreign Application Priority Data
Jul. 4, 2016 (DE) 10 2016 112 179.7

(51) Int. Cl. A61N 1/05

(52) U.S. Cl. CPC A61N 1/057; A61N 2001/0582

(57) ABSTRACT

An electrode fixing sleeve for fixing an electrode lead to biological tissue, the electrode fixing sleeve including a distal sleeve section and a proximal sleeve section, at least one distal electrode guiding region on the distal sleeve section and at least one proximal electrode guiding region on the proximal sleeve section, the distal electrode guiding region and proximal electrode guiding region having a common longitudinal axis. The distal and proximal sleeve sections are adjustable relative to one another along the common longitudinal axis. The electrode fixing sleeve additionally includes at least one electrode fixing element at least parts of which have elastic properties, the electrode fixing element being mounted on both the distal and proximal sleeve sections, and being designed so that at least parts of it move along the common longitudinal axis if a tensile force acts on the electrode fixing element along the common longitudinal axis.

### CROSS-REFERENCE TO RELATED APPLICATIONS

This patent application claims the benefit of and priority to co-pending German Patent Application No. DE 10 2016 112 179.7, filed on Jul. 4, 2016 in the German Patent Office, which is hereby incorporated by reference in its entirety.

### FIELD OF THE INVENTION

Objects of the present invention include, but are not limited to, an electrode fixing sleeve for fixing an electrode lead to biological tissue according to the preamble of claim 1, and a system according to the preamble of claim 11 comprising the electrode fixing sleeve.

### DESCRIPTION OF THE RELATED ART

Cardiovascular diseases are among the most serious diseases of modern society. Even today, many diseases still have a fatal course. Especially older persons are affected by cardiovascular diseases. Therefore, in view of rising life expectancy and the growing number of chronic heart diseases, it can be expected that there will be a further increase in these diseases. The main causes, which are cited again and again, are widespread influencing factors of a modern and globally integrated society such as, for example, stress, smoking, excessively fatty food, and the accompanying overweight or high blood pressure. But genetic predisposition or viral infections can also cause heart diseases. Since cardiovascular system disorders already manifest themselves in the younger years, and especially since younger people are growing up from the very beginning in an environment favoring these influencing factors, a further intensification of the trend toward cardiovascular diseases should be expected. Therefore, an important starting point is a consistent improvement in medical care.

This leads first to rising costs and second also to higher requirements on the safety of medical technology products, for example, cardiac pacemakers, since the future trend is toward an increasing number of these systems being in circulation, and the safety against malfunctions must be correspondingly high.

Non-homogeneous contraction of the individual areas of the left ventricle can cause heart failure, also called cardiac insufficiency. The main reason for non-homogeneous, or asynchronous, contraction is a disturbance in the electrical conduction system of the heart. So-called cardiac resynchronization therapy, CRT stimulation for short, makes the contraction of the left ventricle homogenous again, or simply resynchronizes it, allowing the heart to recover its pumping force again. The process involves the use of two electrodes. One electrode is implanted in the right ventricle. The target vessel for the electrode affecting the left ventricle is the coronary sinus. This is the name of the vessel that carries venous blood from the coronary vessels into the right atrium. The electrode is implanted into a side branch of the coronary sinus, and lies on the outer surface of the left ventricle. Simultaneously delivering pulses through both electrodes once again causes synchronous electrical stimulation of the left part of the heart and allows homogeneous contraction.

The electric leads are fastened to biological tissue through so-called electrode fixing sleeves. This means a special fixing sleeve which is tightly arranged on the electrode lead at a defined place and which is then fastened at a suitable place in the human body, for example, by sewing it on in the area of a muscle or a vessel. Newer variants involve the use of special fixing sleeves that are pressed together or screwed together, and then form a solid unit with the electrode. Such electrode fixing sleeves are frequently made of a soft plastic and are pushed over the electrode lead. Threads, also called ligatures, are then used to apply a fixation force to the electrode fixing sleeve, pressing the latter against the electrode lead. The resulting static friction axially fixes the electrode to the electrode lead. The electrode fixing sleeve can then be sutured at a suitable place in the body.

It turns out to be a problem that manual application of the ligature can damage the electrode lead if the applied fixation force is too high. However, in another case, an unwanted displacement of the electrode lead can occur if the applied fixation force is too small. Furthermore, it turns out to be difficult to correct the position of the electrode lead in the electrode fixing sleeve, if necessary.

European Patent No. 1 933 934 discloses a suture sleeve for fastening an implantable lead to body tissues. This document proposes using a locking mechanism to avoid damage to the electrode lead.

U.S. Pat. No. 4,553,961 also describes a suture sleeve with a gripping enhancing structure that is arranged in the suture sleeve and that has, for example, additional tooth-like structures with which the electrode lead can be securely fixed. The gripping enhancing structure simultaneously provides protection against the ligature exerting too much force on the electrode lead.

U.S. Pat. No. 5,824,032 shows an anchoring sleeve for fastening an electrode lead in human tissue. The electrode lead is fastened in the anchoring sleeve using a kind of tab that is designed to wrap around the electrode lead and secure it by locking.

U.S. Pat. No. 8,271,096 also discloses an electrode fixing sleeve made of an elastic material with a compression governor arranged in it that is adapted to limit compressive forces.

The present invention is directed toward overcoming one or more of the above-mentioned problems.

The present invention now has a goal of creating an alternative electrode fixing sleeve that avoids local damage of the electrode lead by applying a precisely defined, uniform fixation force to it. It should allow the fixation of the electrode lead to be corrected in a flexible manner.

### BRIEF SUMMARY OF THE INVENTION

At least this goal is accomplished by the objects having the features of claims 1 and 11.

A first aspect of the present invention relates to an electrode fixing sleeve for fixing an electrode lead to biological tissue, the electrode fixing sleeve comprising a distal sleeve section and a proximal sleeve section, and at least one distal electrode guiding region on the distal sleeve section and at least one proximal electrode guiding region on the proximal sleeve section, the distal electrode guiding region and proximal electrode guiding region having a common longitudinal axis. The present invention provides that the distal sleeve section and the proximal sleeve section are adjustable relative to one another along the common longitudinal axis, and the electrode fixing sleeve additionally comprises at least one electrode fixing element at least parts of which have elastic properties, the electrode fixing element being mounted both on the distal sleeve section and on the proximal sleeve section, and being designed so that at least parts of it move along the common longitudinal axis if a tensile force acts on the electrode fixing element along the common longitudinal axis.

In other words, an elongation of the electrode fixing element along the common longitudinal axis presses the electrode fixing element against an electrode lead, if the latter is guided along the common longitudinal axis through the distal and proximal electrode guiding regions. Preferably, the pressing is radial with respect to the common longitudinal axis. Furthermore, the radial pressing with respect to the common longitudinal axis is preferably from at least two sides. The electrode fixing element is able, so to speak, to convert the tensile force into a fixation force that can be transferred to the electrode lead. The fixation force is transferable to the electrode lead in the radial direction with respect to the common longitudinal axis. In principle, a fixation principle based on fixation force can be based on a form-fit and/or a frictional connection. A form-fit fixation can be produced, for example, through retaining elements, purely as an example, through small barbs that are provided on an adhesive surface of the electrode fixing element. However, preferably a friction-fit fixation principle is used. For this purpose, it is provided that a frictional engagement can be produced between the adhesive surface of the electrode fixing element and an outer lateral surface of the electrode lead. The frictional engagement can be produced by moving the distal sleeve section and the proximal sleeve section away from one another. As a consequence of this, the electrode fixing element is elongated. The elongation is preferably reversible. This is achieved by the elastic properties of the electrode fixing element. The elongation allows the adhesive surface of the electrode fixing element to be pressed against the electrode lead, if the electrode lead is arranged in the electrode fixing sleeve, so that the frictional engagement is formed by friction. The frictional engagement can be undone by moving the distal sleeve section and the proximal sleeve section toward one another. The electrode fixing element then returns to its original shape, and the elongation is reversed. In this state, the adhesive surface is no longer pressed against the electrode lead and the frictional engagement is undone. The elastic properties of the electrode fixing element can be realized by selecting a suitable structure and/or an elastic material. Possible elastic materials are, for example, elastic plastics or rubbers. If the elastic properties are produced through a structure, it is also possible to use materials without significant elastic properties of their own. Some examples of materials are polyamides, polyurethanes, polyesters, and copolymers of them. Furthermore, thermoplastic polyolefins and polystyrenes are suitable. It is also possible to braid metal wires made of nitinol or highly alloyed cobalt chromium steel (316L, L605) or carbon fibers/Kevlar® fibers in the form of a tube, for example. A suitable basic structure for the electrode fixing element is, for example, a woven fabric, a mesh, or a sheet of a solid material. The basic structure can preferably be spatially shaped into a structure, for example, a tubular structure. For the present invention, it is preferable that the elastic properties be realized by the choice of a suitable structure and an elastic material. For example, if the basic structure is a mesh made of elastic material, a tensile force acting on this mesh leads to an elongation in one direction and to a transverse contraction transverse to this direction. If the mesh is additionally spatially arranged in a structure, for example, in the form of a tube around the common longitudinal axis, elongation of the structure leads to a radial transverse contraction around the common longitudinal axis.

The inventive electrode fixing sleeve offers a series of advantages. For example, the fixation force transferable to the electrode lead through the electrode fixing element can be distributed in an especially homogeneous manner. Since even for elastic components, such as the electrode fixing element, elongation is limited by the material and structure, the transverse contraction that occurs is also limited. This makes it possible to limit the fixation force. In addition, the fixation force that can be applied to the electrode lead can be precisely defined, since there is a systematic correlation between the fixation force and the elongation of the electrode fixing element. All this has the result that it is possible to produce a secure frictional engagement between the electrode lead and the electrode guiding region, and to avoid too strong a strain on the electrode lead. In addition, the fixation force that is transferable onto the electrode lead is reversible.

A preferred embodiment of the inventive electrode fixing sleeve provides that the electrode fixing element has at least one adhesive surface that faces the common longitudinal axis and that has a coefficient of friction of at least 0.8 with respect to plastics. However, the values can also be above or below this. Preferably, the plastic specifically considered for designing the adhesive surface is a material that can be used for a sheath of an electrode lead. Some examples here are silicones, polyurethanes, and copolymers of them, as well as copolyamides (PEBAX). Other examples are styrene block copolymers (SBS, SEBS, SEPS, SEEPS, MBS), olefin-based elastomers, and thermoplastic polyesters. This offers the advantage that the frictional engagement between the electrode lead and the adhesive surface of the electrode fixing element can be realized in an especially simple and reliable manner.

Another preferred embodiment of the inventive electrode fixing sleeve provides that the electrode fixing element comprises a tubular structure whose longitudinal axis runs collinear to the common longitudinal axis.

The tubular structure is especially suitable to convert an elongation as a consequence of the tensile force into a transverse contraction to apply the fixation force. Preferably, a sheet of a solid elastic material is selected as the basic structure for the tubular structure. The tubular structure made of an elastic material is also directly available in the form of a semi-finished product.

Another preferred embodiment of the inventive electrode fixing sleeve provides that the tubular structure has a defined transverse contraction behavior, if the tubular structure experiences a defined elongation or longitudinal expansion.

The tubular structure reduces its diameter, so to speak, when there is a transverse contraction. The transverse contraction behavior is designed using the ratio of the reduction in the diameter of the tubular structure divided by its longitudinal expansion. A maximum longitudinal expansion of 20 mm preferably leads to a maximum reduction of 4 mm in diameter. In this case, the ratio is 5. Preferably, the ratio lies in the range from 2 to 4.75, more preferably from 4 to 4.5. Since elastic material properties and structural properties are frequently nonlinear in practice, in such cases it is expedient to use a linear approximation of the ratio. For the person skilled in the art, it is clear without a second thought that the design must be theoretically based on a specific electrode lead. A linear approximation of the ratio is then made for a range of longitudinal expansion values within which there is a transition point from the absence of a frictional engagement to the presence of a frictional engagement.

Another preferred embodiment of the inventive electrode fixing sleeve provides that the adhesive surface of the electrode fixing element be 30 mm² to 200 mm². It is further preferred for the adhesive surface of the electrode fixing element to be 50 mm² to 150 mm², further preferred 70 mm² to 120 mm², and further preferred 90 mm² to 100 mm².

The required longitudinal expansion of the electrode fixing element affects the overall size of the electrode fixing sleeve, especially its overall length. Since the electrode fixing sleeve is placed in the human body, it is necessary for this length to be limited. Investigations have shown that these orders of magnitude of the adhesive surface ensure a more secure frictional engagement without requiring too large a longitudinal expansion of the electrode fixing element.

Observing the above design parameters advantageously makes it possible to limit the maximum overall length of the electrode fixing sleeve to 20 mm to 30 mm. The overall length of the electrode fixing sleeve is preferably 23 mm to 27 mm.

Another preferred embodiment of the inventive electrode fixing sleeve provides that the electrode fixing element comprises, at a distal end and/or at a proximal end, at least one sliding body with which the electrode fixing element is mounted on the proximal sleeve section and/or the distal sleeve section so that it can rotate around the common longitudinal axis and be fixed along the common longitudinal axis.

Each of the sliding bodies can be, for example, a washer that is mounted in a radial receiving groove in the proximal sleeve section and the distal sleeve section. This allows the electrode fixing element to rotate freely around the common longitudinal axis. Of course, the electrode fixing element can also have a sliding body only on one side, and on the other side be solidly connected with the proximal or distal sleeve section.

This advantageously ensures that no unwanted transverse contraction of the electrode fixing element occurs as a consequence of an unwanted twisting.

Another preferred embodiment of the inventive electrode fixing sleeve provides that at least sections of the electrode fixing element be arranged in the distal electrode guiding region and the proximal electrode guiding region.

This offers the advantage that an area for transfer of the fixation force to the electrode lead is enlarged.

Another preferred embodiment of the inventive electrode fixing sleeve provides that the proximal sleeve section and the distal sleeve section be adjustable relative to one another along the common longitudinal axis through a thread.

This offers the advantage that the proximal sleeve section and the distal sleeve section can be moved away from one another or toward one another in an especially simple and exact way. In addition, the electrode fixing element can be elongated very exactly and with little manual force. This embodiment is especially advantageous using one sliding body at the distal end and one at the proximal end of the electrode fixing element to compensate for rotation of the thread.

Another preferred embodiment of the inventive electrode fixing sleeve provides that the electrode fixing sleeve comprises a securing element to secure a relative adjustment state of the distal sleeve section and the proximal sleeve section. The adjustment state comprises at least a separation of the distal sleeve section and the proximal sleeve section along the common longitudinal axis. It can also comprise their relative orientation around the common longitudinal axis.

The securing element advantageously ensures that an adjusted longitudinal expansion of the electrode fixing element remains secure. The securing element can also serve as a mark showing when the desired adjustment state is reached. This ensures that the fixation force is not incorrectly adjusted.

Another preferred embodiment of the inventive electrode fixing sleeve provides that the electrode fixing sleeve comprises a securing element in the form of a thread interruption and/or a catch.

These are especially simple and reliable ways of realizing the securing element.

A second aspect of the present invention relates to a system comprising at least one inventive electrode fixing sleeve and one electrode lead.

The electrode fixing sleeve of the inventive system and the electrode lead according to the above description. This also applies for the above-described structural connections and interactions that can be realized between the electrode fixing sleeve and the electrode lead.

The inventive system offers the advantage that the electrode fixing element of the electrode fixing sleeve makes it possible to transfer a defined fixation force onto the electrode lead in a homogeneous and reliable manner. All other advantages result in detail from the above description.

A preferred embodiment of the inventive system provides that the coefficient of friction between the electrode fixing element of the electrode fixing sleeve and the electrode lead be at least 0.8.

This offers the advantage that the frictional engagement between the electrode lead and the electrode fixing element can be realized in an especially simple and reliable manner.

Another preferred embodiment of the inventive system provides that relative adjustment of the distal sleeve section and the proximal sleeve section along the common longitudinal axis can produce a corresponding longitudinal expansion of the tubular structure of the electrode fixing element and, as a consequence of the defined transverse contraction behavior of the tubular structure, create the frictional engagement between the tubular structure and the electrode lead.

For instance, a defined frictional engagement between the electrode lead and the tubular structure can be created in an advantageously simple and reliable manner.

A preferred embodiment of the inventive system provides that the frictional engagement is able to transfer a static friction of from 2 N to 50 N. It is preferred for the transferable static friction to be from 5 N to 25 N, further preferred from 7 N to 10 N.

A preferred embodiment of the inventive system provides that the frictional engagement can be produced by a relative adjustment travel of the distal sleeve section and the proximal sleeve section of from 2 mm to 20 mm. The adjustment travel is preferably from 5 mm to 15 mm, more preferably from 7 mm to 10 mm.

This advantageously makes it possible for the electrode fixing sleeve to be compact in size and for the frictional engagement to be secure.

Further embodiments, objectives, features, advantages and possible applications of the present invention will become clear from the following description of exemplary embodiments with reference to the drawings. Here, all features described and/or illustrated in the drawings form the subject matter of the present invention, independently or in any combination, even regardless of their summary in the claims and the dependency references of the claims.

### BRIEF DESCRIPTION OF THE DRAWINGS

The above and other aspects, features and advantages of at least one embodiment of the present invention will be more apparent from the following more particular description thereof, presented in conjunction with the following drawings, wherein:

FIG. 1 shows a preferred embodiment of an inventive system with an inventive electrode fixing sleeve and an electrode lead.

### DETAILED DESCRIPTION OF THE INVENTION

The following description is of the best mode presently contemplated for carrying out at least one embodiment of the present invention. This description is not to be taken in a limiting sense, but is made merely for the purpose of describing the general principles of the preset invention. The scope of the present invention should be determined with reference to the claims.

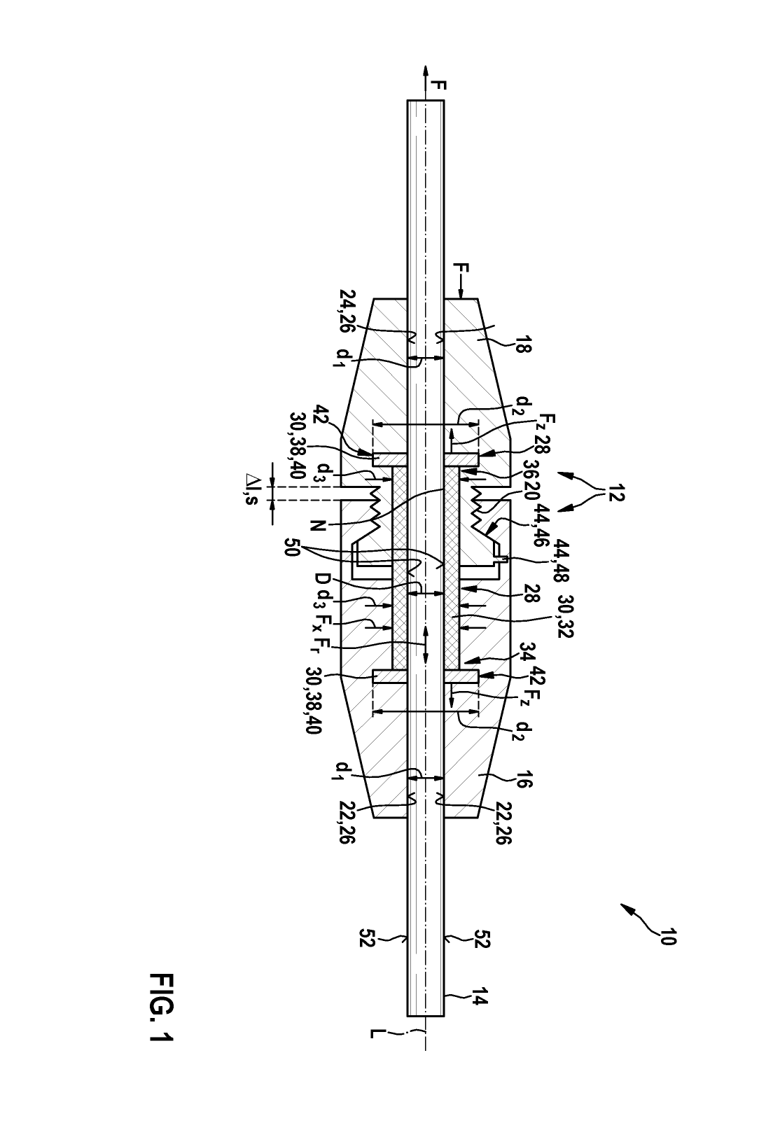

FIG. 1 shows an inventive system 10 with an inventive electrode fixing sleeve 12 and an electrode lead 14. The electrode fixing sleeve 12 is shown in a cross-sectional view.

The inventive electrode fixing sleeve 12 has a distal sleeve section 16 and a proximal sleeve section 18. The proximal sleeve section 18 and the distal sleeve section 16 are adjustable relative to one another along the common longitudinal axis L through a thread 20. The distal sleeve section 16 has a distal electrode guiding region 22, and the proximal sleeve section 18 has a proximal electrode guiding region 24. The proximal and distal electrode guiding regions 22, 24 are each in the form of cylindrical holes 26 through the respective sleeve sections 16, 18. A diameter d1-3 of the cylindrical through holes 26 varies along the common longitudinal axis L. This produces a receiving area 28 for an electrode fixing element 30. The electrode fixing element 30 comprises a tubular structure 32, which has one sliding body 38 each at a distal end 34 and a proximal end 36. Here the respective sliding bodies 38 are in the form of washers 40. The washers 40 are arranged in areas of the cylindrical through holes 26, which have the diameter d2 and thus each form a receiving groove 42 for the washers 40. Each receiving groove 42 has one of the washers 40 mounted in it so that it can rotate about the common longitudinal axis L. The tubular structure 32 is arranged in areas of the cylindrical through holes 26 which have the diameter d3. The washers 40 also allow the tubular structure 32 to rotate about the common longitudinal axis L. The tubular structure 32 consists of silicone and has elastic material properties. The tubular structure 32 here is also in the form of a woven fabric. Generally speaking, the tubular structure 32 here functions according to the principle of the so-called Chinese finger trap.

Adjustment of the distal sleeve section 16 and the proximal sleeve section 18 relative to one another along the common longitudinal axis L through the thread 20 can apply a tensile force Fz onto the tubular structure 32 through the washers 40. This causes the tubular structure 32 to undergo a longitudinal expansion Δl. The longitudinal expansion Δl also corresponds to an adjustment travel, covered by the distal sleeve section 16 relative to the proximal sleeve section 18, along the common longitudinal axis L. Here the longitudinal expansion Δl or the adjustment travel are limited by a securing element 44 in the form of a thread interruption 46. The securing element additionally comprises a catch 48, which ensures that the distal sleeve section 16 and the proximal sleeve section 18 remain at a defined adjustment travel s corresponding to a defined relative adjustment state. The tubular structure 32 has defined transverse contraction behavior. The longitudinal expansion Δl elastically reduces a diameter D of the tubular structure 32.

In the inventive system 10, which also comprises, in addition to the electrode fixing sleeve 12, the electrode lead 14 as an element, the reduction of the diameter D presses an adhesive surface 50 of the tubular structure 32 against an outer lateral surface 52 of the electrode lead 14. This applies a fixation force Fx onto the electrode lead 14. This leads to a frictional engagement between the outer lateral surface 52 of the electrode lead 14 and the adhesive surface 50 of the tubular structure 32.

In this case, an adjustment travel or a longitudinal expansion Δl of 3 mm reduces the diameter D by 0.6 mm. These values give a value of 20 N for the fixation force Fx. Here the adhesive surface 50 of the tubular structure 32 is 100 mm², so that the fixation force Fx leads to a surface pressure of 0.2 N/mm². In this case, the material used for the tubular structure 32 is a woven fabric made of polyester, polyamide, nylon, or Kevlar®. A mixed woven fabric is also possible. The outer sheath 52 of the electrode lead 14 consists of silicone or silicone-urethane-copolymer. Thus, between the adhesive surface 50 of the tubular structure 32 and the outer sheath 52 of the electrode lead 14 there is a coefficient of friction μ of 0.8 through 1.2. However, the coefficient of friction μ can also be above it or below it, and can be influenced, for example, by applying a lubricant. The competent person skilled in the art determines this on the basis of the disclosed design aspects. As a consequence this, the frictional engagement is reliably able to keep the electrode lead 14 from slipping in the electrode fixing sleeve 12 up to a force F of 20 N acting on the electrode lead 14.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention. Additionally, the disclosure of a range of values is a disclosure of every numerical value within that range.

I/We claim:

1. An electrode fixing sleeve for fixing an electrode lead to biological tissue, comprising:
a distal sleeve section and a proximal sleeve section;
at least one distal electrode guiding region on the distal sleeve section and at least one proximal electrode guiding region on the proximal sleeve section; and
a common longitudinal axis of the distal electrode guiding region and the proximal electrode guiding region,
wherein the distal sleeve section and the proximal sleeve section are adjustable relative to one another along the common longitudinal axis, and the electrode fixing sleeve additionally comprises at least one electrode fixing element at least parts of which have elastic properties, the electrode fixing element being mounted both on the distal sleeve section and on the proximal sleeve section, and being designed so that at least parts of it move along the common longitudinal axis if a tensile force acts on the electrode fixing element along the common longitudinal axis.

2. The electrode fixing sleeve according to claim 1, wherein the electrode fixing element has at least one adhesive surface that faces the common longitudinal axis and that has a coefficient of friction of at least 0.8 with respect to plastics.

3. The electrode fixing sleeve according to claim 1, wherein the electrode fixing element comprises a tubular structure, whose longitudinal axis runs collinear with the common longitudinal axis.

4. The electrode fixing sleeve according to claim 3, wherein the tubular structure has a defined transverse contraction behavior, if the tubular structure undergoes a defined longitudinal expansion.

5. The electrode fixing sleeve according to claim 1, wherein the adhesive surface of the electrode fixing element is 30 mm² to 200 mm².

6. The electrode fixing sleeve according to claim 1, wherein the electrode fixing element comprises, at a distal end and/or at a proximal end, at least one sliding body with which the electrode fixing element is mounted on the proximal sleeve section and/or the distal sleeve section so that it can rotate about the common longitudinal axis and be fixed along the common longitudinal axis.

7. The electrode fixing sleeve according to claim 1, wherein at least sections of the electrode fixing element are arranged in the distal electrode guiding region and the proximal electrode guiding region.

8. The electrode fixing sleeve according to claim 1, wherein the proximal sleeve section and the distal sleeve section are adjustable relative to one another along the common longitudinal axis through a thread.

9. The electrode fixing sleeve according to claim 1, wherein the electrode fixing sleeve comprises a securing element to secure a relative adjustment state of the distal sleeve section and the proximal sleeve section.

10. The electrode fixing sleeve according to claim 8, wherein the electrode fixing sleeve comprises a securing element in the form of a thread interruption and/or a catch.

11. A system comprising at least:
an electrode fixing sleeve according to claim 1; and
an electrode lead.

12. The system according to claim 11, wherein a coefficient of friction between the electrode fixing element of the electrode fixing sleeve and the electrode lead is at least 0.8.

13. The system according to claim 11, wherein relative adjustment of the distal sleeve section and the proximal sleeve section along the common longitudinal axis can produce a corresponding longitudinal expansion of a tubular structure of the electrode fixing element and, as a consequence of a defined transverse contraction behavior of the tubular structure, create a frictional engagement between the tubular structure and the electrode lead.

14. The system according to claim 13, wherein the frictional engagement is able to transfer a static friction of from 2 N to 50 N.

15. The system according to claim 14, wherein the frictional engagement is produced by a relative adjustment travel of from 2 mm to 20 mm.
